(19) United States
(12) Patent Application Publication
(10) Pub. No.: US 2023/0019703 A1
(43) Pub. Date: Jan. 19, 2023

(54) PERSONAL HEALTH TRACKING URINALYSIS DEVICE

(71) Applicants: Babak Behzad, Irvine, CA (US); Banafsheh Behzad, Orange, CA (US); Elmira Amiri Souri, London (GB); Hadi Tavassol, Orange, CA (US)

(72) Inventors: Babak Behzad, Irvine, CA (US); Banafsheh Behzad, Orange, CA (US); Elmira Amiri Souri, London (GB); Hadi Tavassol, Orange, CA (US)

(21) Appl. No.: 17/861,370

(22) Filed: Jul. 11, 2022

Related U.S. Application Data
(60) Provisional application No. 63/219,935, filed on Jul. 9, 2021.

(51) Int. Cl. A61B 5/20; A61B 5/1172; A61B 5/00

(52) U.S. Cl. CPC A61B 5/208; A61B 5/1172; A61B 5/749; A61B 5/746; A61B 5/7275

(57) ABSTRACT

A health monitoring urinalysis system that monitors several health indicators from human urine is provided. The system includes a temperature sensor and several electrochemical sensors that are installed in a urine collection basin, such as, a toilet or a urinal and may automatically collect urine information after each use. The system collects data during the routine and normal use of a urine collection basin. The system includes a control and measurement unit that may be installed outside the urine collection basin. The control and measurement unit receives sensor measurements and transmits the measurements to one or more remote electronic devices. The remote electronic devices and/or the processor of the control and measurement unit perform data analysis, provide diagnostic, and generate health alerts. The system performs recurrent health monitoring after the urine collection basin is used and may detect abnormal conditions at early stages, in addition to a routine urine test.

### CLAIM OF BENEFIT TO PRIOR APPLICATIONS

This application claims the benefit of U.S. Provisional Patent Application Ser. No. 63/219,935, filed on Jul. 9, 2021. The contents of U.S. Provisional Patent Application 63/219,935 are hereby incorporated by reference.

### BACKGROUND

Personal health monitoring devices (PHMD) are systems that monitor an individual's health behavior over time. These devices have two main characteristics: they collect and transfer data to the cloud to perform analyses, and they may be used by the individuals outside of clinical environments. Among PHMDs are wearable health devices, which facilitate constant monitoring of an individual's health condition. Wearable health devices are used to constantly monitor a person's vital signs.

### BRIEF DESCRIPTION OF THE DRAWINGS

The various embodiments of the present personal health tracking urinalysis device now will be discussed in detail with an emphasis on highlighting the advantageous features. These embodiments depict the novel and non-obvious personal health tracking urinalysis device shown in the accompanying drawings, which are for illustrative purposes only. These drawings include the following figures, in which like numerals indicate like parts:

FIG. 1 is a functional block diagram illustrating an example health monitoring urinalysis system for recurrent urinalysis, tracking health condition, and performing disease prediction, according to various aspects of the present disclosure;

FIG. 2A is a schematic front view of a circuit card that includes a plurality of sensors, according to various aspects of the present disclosure;

FIG. 2B is a schematic front view of a plurality of circuit cards that each include one or more sensors, according to various aspects of the present disclosure;

FIG. 3 is a perspective view of a urine collection basin and a personal health monitoring device, according to various aspects of the present disclosure;

FIG. 4 is a functional block diagram illustrating a housing for the sensors, according to various aspects of the present disclosure;

FIG. 5A illustrates an example of the responses of three electrochemical sensors from the analysis of a midday urine, according to various aspects of the present embodiments;

FIG. 5B identifies the portion of the response of the sensor with glassy carbon electrodes of FIG. 5A that relates to dopamine, according to various aspects of the present disclosure;

FIG. 5C illustrates graphs showing the response from a set of dopamine reference solutions with increasing concentration, according to various aspects of the present disclosure;

FIG. 5D illustrates a graph that shows the peak currents of a set of reference solutions at particular voltages;

FIG. 6 is a functional block diagram illustrating an example health monitoring urinalysis system with several groups of sensors installed inside different urine collection basins at different locations and the corresponding control and management units, according to various aspects of the present disclosure;

FIG. 7 is a functional diagram illustrating an example platform for recurrent urinalysis and developing a machine learning model to infer health insights, according to various aspects of the present disclosure;

FIG. 8 is a functional diagram illustrating an example data infrastructure used for handling data flow, according to various aspects of the present disclosure;

FIG. 9 is a front perspective of a sample container that includes a group of sensors, according to various aspects of the present disclosure;

FIG. 10 is a flowchart illustrating an example process for collecting data by a PHMD, according to various aspects of the present disclosure;

FIG. 11 illustrates the response from the sensors represented as current amplitude maps, pH trends, and temperature trends, according to various aspects of the present disclosure;

FIG. 12 illustrates example results of XGBoost model, where 80% of the data is used for training, 10% of the data is used for validation and hyperparameter tuning, and 10% of the data is used for unseen test data, according to various aspects of the present disclosure; and

FIG. 13 is a functional block diagram illustrating an example electronic system, according to various aspects of the present disclosure.

### DETAILED DESCRIPTION

One aspect of the present embodiments includes the realization that the existing PHMDs are not capable of monitoring biomarkers from inner workings of human body, which report on many health conditions. Furthermore, the wearable PHMDs, in many cases, are not convenient to wear constantly. The use of gas chromatography and mass spectrometry (GC-MS) have been proposed in a personalized platform. However, extracting hundreds of urine metabolites from a urine sample may not be practical, as mass spectrometry can only be done in the labs and may not be practical for widespread use. A personal toilet with a gas chromatography and mass spectrometry mounted in it may not be practical or reasonable.

Furthermore, a mountable toilet system has been proposed for doing urine and stool analysis for personalized health monitoring. This system uses motion sensors, cameras, colorimetric assay, and computer vision. Privacy is a major concern with this approach, as there are multiple built-in cameras in this proposed smart toilet.

The present embodiments, as described in detail below, solve the above-mentioned problems by providing a health monitoring urinalysis system that monitors several health indicators from human urine. The health monitoring urinalysis system may include several sensors that may be installed in a urine collection basin, such as, a toilet or a urinal and may automatically collect urine information after each use. The health monitoring urinalysis system of the present embodiments provides the technical advantage of collecting data during the routine and normal use of a urine collection basin and does not cause any discomfort or interference with one's daily routines.

The health monitoring urinalysis system of the present embodiments may include a control and measurement unit that may be installed outside the urine collection basin. The control and measurement unit and the sensors may be communicatively coupled, for example, by a set of wires. The control and measurement unit may receive sensor measurements and may transmit the measurements to one or more remote electronic devices through one or more networks, such as the Internet. The remote electronic devices and/or the processor of the control and measurement unit may perform data analysis, provide diagnostic, generate health alerts, etc.

One of the technical advantages provided by the health monitoring urinalysis system of the present embodiments is performing recurrent health monitoring (e.g., performed several times a day when the urine collection basin is used) that may detect abnormal conditions at early stages, in addition to a routine urine test. The health monitoring urinalysis system of the present embodiments may be used to collect urine data and perform health monitoring and health alert generation for several persons that may use the same urine collection basin. The remote electronic devices may collect data measured from different groups of sensors that are installed inside different urine collection basins at different locations. By collecting large-scale data over time from many individuals, the present embodiments may provide personalized health insights for the risk of developing serious health conditions, and/or may provide preventive recommendations.

The health monitoring urinalysis system of the present embodiments may constantly monitor the inner workings of human body, through the repetitive measuring of urine's pH, temperature, metabolic information such as minerals, proteins, glucose, uric acid, vitamins, and biomarkers related to ovulation, pregnancy, and urinary tract infection, among others. Some embodiments may use artificial intelligence (AI) or machine learning (ML) to perform statistical analysis of key metabolites and features related to health conditions from many (electrochemical) sensors across many individuals to identify health patterns.

In addition to the urinalysis of the target metabolites, the collected large-scale data and machine learning may assist with detection of trends, which may cause serious health conditions, including chronic diseases, such as, cancer, heart diseases, diabetes, kidney diseases, neurological diseases, obesity, and mental disorders. Constant monitoring of urine temperature, pH, and key metabolites provides the technical advantage of predicting and monitoring physiological changes such as those related to epidemic and pandemics of viral infections (e.g., coronaviruses).

The remaining detailed description describes the present embodiments with reference to the drawings. In the drawings, reference numbers label elements of the present embodiments. These reference numbers are reproduced below in connection with the discussion of the corresponding drawing features.

Urine is a rich and complex mixture, which is known to report on more than 4000 metabolites and approximately 600 human conditions. Urine metabolite reflects and fluctuates with individual health and habits very closely, as it forms through filtration of an estimated 200 liters of blood daily and is not subject to homeostasis mechanisms. Hence, many physiological conditions such as gender, age, diet, daily routine, sleep patterns, exercise, hormone cycles, pregnancy, biomarker changes due to development of diseases, presence of pathogens, drug use, temperature changes (fever), and pH changes (diet and infections) may be directly reflected in urine.

Irregularities and changes in the composition of urine metabolite may point to changes in personal routine and/or physical, mental and neurological conditions, and behavioral changes. Urine metabolites have been linked to cancer, obesity, inflammation, neurological diseases, and mental disorders.

Urine is an aqueous mixture of mainly water, urea, creatinine, chloride, sodium, potassium along with many compounds with high regular contents such as hippuric acid, citric acid, uric acid, D-glucose, amino acids (e.g., glycine, L-cysteine, and L-glutamine), and amines (e.g., trimethylamine N-oxide and ethanolamine). Urine may also indicate consumption of drugs, pain killers, substance abuse, alcohol consumption and vitamins (e.g., ascorbic acid (vitamin C) and riboflavin (vitamin B2)). However, such a rich mixture makes constant monitoring of urine outside clinical setups impractical. Hence the accessible device of the present embodiments provides the technical advantage of a small footprint capable of real time analysis without requiring sample separation or pretreatment.

### I. Health Monitoring Urinalysis System

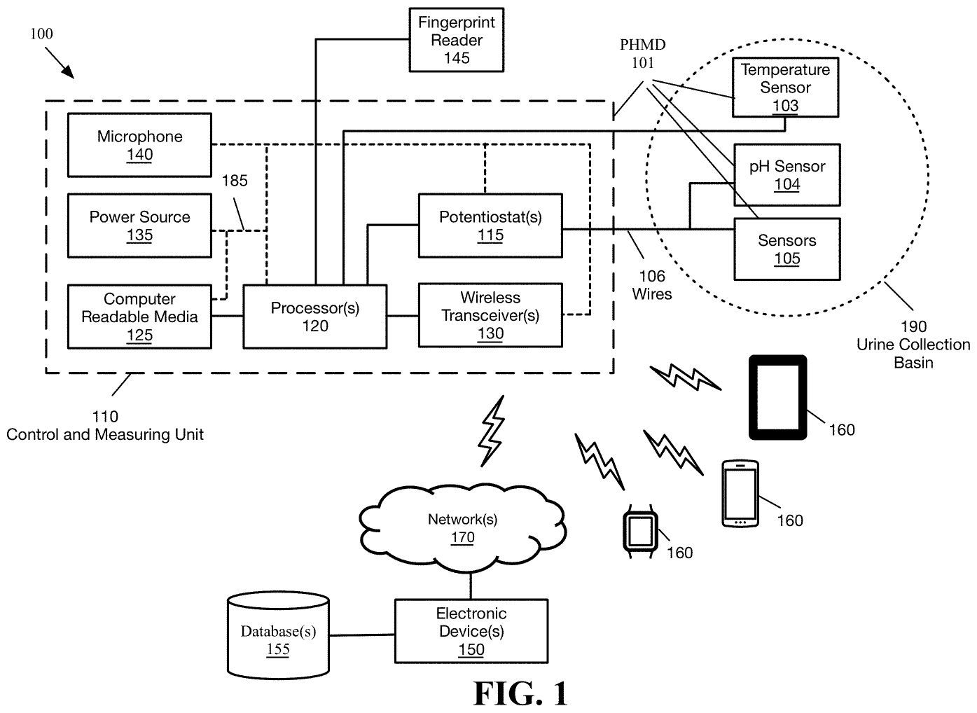

Some of the present embodiments provide a health monitoring urinalysis system that is configured to track health conditions and perform disease prediction for one or more persons. FIG. 1 is a functional block diagram illustrating an example health monitoring urinalysis system 100 for recurrent urinalysis, tracking health condition, and performing disease prediction, according to various aspects of the present disclosure. With reference to FIG. 1, the health monitoring urinalysis system 100 may include a PHMD 101, one or more electronic devices 150, one or more databases 155, and/or one or more client devices 160. The PHMD 101 may include a plurality of sensors 103-105 and a control and measuring unit 110.

The sensors may include a temperature sensor 103, a pH sensor 104, and one or more other sensors 105. The pH sensor 104 and the sensors 105, in some embodiments, may be electrochemical sensors. Each sensor 103-105 may include a plurality (e.g., two or three) electrodes. The electrodes may include platinum (Pt) electrodes, gold (Au) electrodes, glassy carbon electrodes, amorphous carbon electrodes, or silver (Ag) electrodes. Glassy carbon (also referred to as glass-like carbon or vitreous carbon) is a non-graphitizable carbon that includes glassy and ceramic properties and graphite properties. Amorphous carbon is the free, reactive carbon that does not have a crystalline structure. The material on the electrodes of each type of electrochemical sensor may better interact with one or more different type of analytes in urine. For example, an electrochemical sensor with a glassy carbon electrode may be more sensitive to dopamine in urine than other type of sensors. The use of multiple different types of electrochemical sensors in the system 100 of present embodiments provides the technical advantage of measuring concentration of different types of analytes in urine by different types of electrochemical sensors.

The sensors electrodes, in some embodiments, may be screen-printed electrodes (SPE). The SPEs are electrochemical devices that are manufactured by printing different types of ink (e.g., carbon, silver, gold, platinum) on plastic or ceramic substrates allowing quick in-situ analysis with high reproducibility, sensitivity and accuracy. The composition of the different inks used in the manufacture of the electrode determines the electrode's selectivity and sensitivity. Accordingly, the electrochemical sensors of the present embodiments may be designed according to the type of analytes and markers that are to be measured in urine. The sensors 104-105, in some embodiments, may include single-walled carbon nanotubes (SWCNT) sensors, multi-walled carbon nanotubes (MWCNT) sensors, NiOₓ (Nickel Oxide based materials) SPEs sensors, sodium ion selective electrochemical sensors, potassium ion selective electrochemical sensors, and/or chloride ion selective electrochemical sensors.

The sensors 105 may measure electrical and/or electrochemical responses of urine to changes in a voltage applied to the electrodes. The type and the number of sensors 105 used in different embodiments may be customizable based on a particular application, and may depend on the type of chemicals or markers that are required to be detected in the urine for a particular application. For example, and without limitations, depending on specific applications, one or more of the sensors may be used to measure and/or detect leukocytes, nitrite, urobilinogen, protein, blood/hemoglobin, S.G, ketones, bilirubin, glucose, prostate-specific antigen (PSA), sodium, leukocyte esterase (LE), red blood cells (RBCs), white blood cells (WBCs), epithelial cells, bacteria, yeast, cholesterol, phosphate, calcium, vitamins, tyrosine, leucine, cystine, cortisol, metabolites, oxalate, estrogen, progesterone, testosterone, adenosine, melatonin, etc.

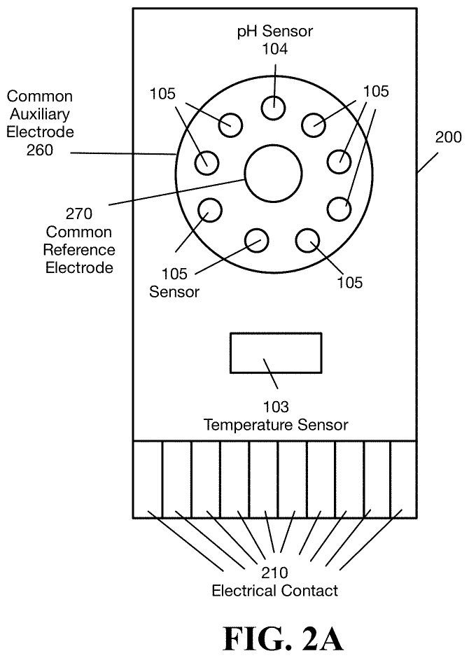

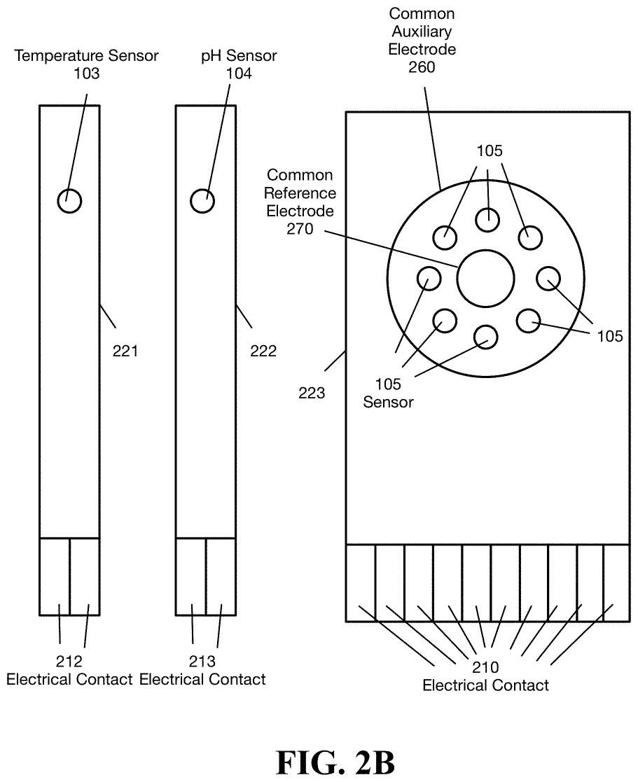

Some of all of the sensors 103-105, in some embodiments, may be placed on one or more circuit cards. FIG. 2A is a schematic front view of a circuit card 200 that includes a plurality of sensors, according to various aspects of the present disclosure. With reference to FIG. 2A, the circuit card 200 may include a temperature sensor 103, a pH sensor 104, and several electrochemical sensors 105.

As described below, electrochemical sensors may include three electrodes: working electrode, reference electrode, and auxiliary electrode. In the example of FIG. 2A, each sensor 104-105 has a separate working electrode. The reference electrode 260 and the auxiliary electrode 270 are common between the sensors 104-105 of FIG. 2A.

Some or all of the sensors 103-105, in some embodiments, may be on separate circuit cards. FIG. 2B is a schematic front view of a plurality of circuit cards that each include one or more sensors, according to various aspects of the present disclosure. In the example of FIG. 2B, the temperature sensor 103 is on the circuit card 221, the Ph sensor 104 is on the circuit card 222, and the sensors 105 are on the circuit card 223. Each sensor 105 in FIG. 2B has a separate working electrode. The reference electrode 260 and the auxiliary electrode 270 are common between the sensors 105 of FIG. 2B.

The card 200 several electrical contacts 210 to carry signals between the sensors 103-105 and different components of the PHMD device 110 such the potentiostat(s) 115 and/or the processor(s) 120. The electrical contacts 210, in some embodiments, may be connected to the potentiostat(s) 115 and/or the processor(s) 120 by a plurality of wires through a card connector, such as a card edge connector. In other embodiments, the electrical contacts 210 may be soldered to a plurality of wires that are connected to the potentiostat(s) 115 and/or the processor(s) 120. Some embodiments may insulate the electrical contacts 210 and/or the card edge connector by a substance such as epoxy to prevent rust formation from exposure to the liquid inside a urine collection basin.

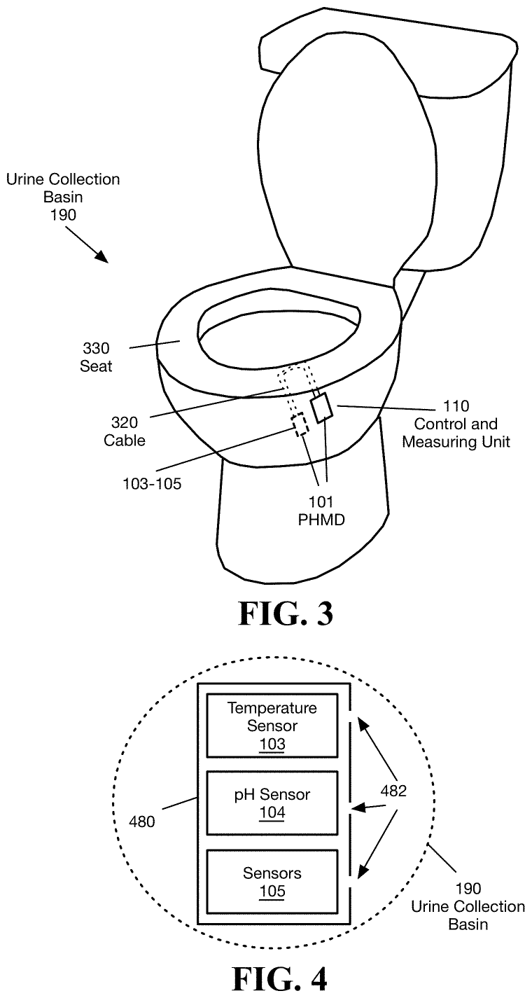

The sensors 103-105 may be installed inside a urine collection basin 190. FIG. 3 is a perspective view of a urine collection basin 190 and a personal health monitoring device 101, according to various aspects of the present disclosure. With reference to FIG. 3, the PHMD 101 may include a control and measurement unit 110 and a plurality of sensors 103-105 (shown by dashed line as the sensors 103-105 are positioned inside the urine collection basin in the perspective view of FIG. 3). The control and measurement unit 110 may be installed on the outside surface of a urine collection basin 190 in a residence, a hospital, a nursing home, etc. The urine collection basin 190 in this example is a toilet bowl.

With further reference to FIG. 3, the control and measurement unit 110 may be connected to the sensors 103-105 by a cable 320 that may include a plurality of wires that connect the sensors 103-105 to the components of the control and measurement unit 110 such as the potentiostat(s) 115 and/or the processor(s) 120. The cable 320 may be posited under the seat 330 of the urine collection basin 190. The sensors 103-105, in some embodiments, may be on one or more circuit cards (as shown by the circuit card 200 of FIG. 2A and circuits cards 221-223 of FIG. 2B). The length of the cable 320 and the position of the control and measurement unit 110 may be configured such that the sensors 103-105 come into contact with water and urine inside the urine collection basin 190. The control and measurement unit 110 may be connected to the outside of the urine collection basin 190, for example, and without limitations, by one or more suction cups.

It should be noted that, some embodiments may connect the sensors 103-105 by the cable 320 to a wireless transceiver positioned on the outside of the urine collection basin 190. These embodiments may position the control and measurement unit 110 within the range of the wireless transceiver that is positioned on the outside of the urine collection basin 190 (e.g., in a close by room or in a closet). The control and measurement unit 110 may communicate with the wireless transceiver that is positioned on the outside of the urine collection basin 190 through the wireless transceiver(s) 130.

In some embodiments, the sensors 103-105 may be at least partially inside a housing to facilitate handling of the sensors. FIG. 4 is a functional block diagram illustrating a housing for the sensors, according to various aspects of the present disclosure. With reference to FIG. 4, the housing 480 may include one or more holes 482 to allow water and urine from the urine collection basin 190 to come into contact with the sensors 103-105. The housing 480 may be installed inside a toilet bowl, a urinal, etc., for example, and without limitations, with one or more suction cups. The housing 180, in some embodiments, may be installed inside the urine collection basin 190 such that the sensors 103-105 may be in contact with water.

The use of the electrochemical sensors by the present embodiments to measure the concentration of different analytes or markers in urine provides several technical advantages. Urine is an electrolyte environment where the ions may move. The electrochemical sensors are sensitive to the content of any liquid that includes free ions. The electrochemical sensors are, therefore, very sensitive to the content of urine and provide large signals for measuring the analytes and biomarkers in the urine. The electrochemical sensors provide further advantage of producing quantitative measurements for the contents of urine. The quantitative values (e.g., digital values) provided by the electrochemical sensors may be related to the concentration of the analytes and markers in the urine. In contrast, the prior art test strips provide visual results based on the change in the color of the strip after applying a quantity of urine to the test strip.

The control and measurement unit 110 may include one or more potentiostat(s) 115, one or more processors 120, computer readable media 125, one or more wireless transceivers 130, and/or a power source. The control and measurement unit 110 may be installed outside of the urine collection basin 190. The control and measurement unit 110, in some embodiments, may be connected to the sensors 103-105 by a set of wires 106, that may be used by the potentiostat(s) 115 to apply voltage (or current) to the sensors' 105 electrodes and/or to read current (or voltage) from the sensors 105. The wires 106 may also be used to read the measurements from temperature sensor 103 and the pH sensor by the processor(s) 120. The control and measurement unit 110 and the sensors 103-105 may be easily removed from the urine collection basins 190.

Each potentiostat 115 may be configured to control one or more of the sensors 105. A sensor 105 with three electrodes may include a working electrode, a reference electrode, and an auxiliary electrode. A potentiostat 115 may control a three-electrode sensor by applying a voltage between the working electrode and the reference electrode, and measuring the current at the auxiliary electrode. The potentiostat 115 may change the voltage and may measure the resulting current to provide pairs of voltage and current values to the processor(s) 120. For sensors with two electrodes, a voltage may be applied between the working and auxiliary electrodes and the current flowing between the two electrodes may be measured and read by the potentiostat 115. It should be noted that, instead of potentiostats, some embodiments may use galvanostats to achieve similar results. A galvanostat applies a current between two points and measures the resulting voltage.

The processor(s) 120 may receive pairs of voltage and current values from the potentiostat(s) 115. For example, a pair of voltage and current values may include the voltage applied by a potentiostat 115 between the working and reference electrodes of a sensor 105 and the current measured at the auxiliary electrode of the sensor.

For each particular application of the health monitoring urinalysis system 100, the potentiostat(s) 115 may provide a set of voltages to each sensor 105 and may measure the resulting current. These voltages are sent by the processor(s) 120 as commands to the potentiostat(s) 115. The processor(s) 120 may receive the voltage and current value pairs from the potentiostat(s) 115 and may send the values of the voltage and current value pairs to the electronic devices 150 through the wireless transceiver(s) 130 and one or more networks 170.

In some embodiments, the electronic devices 150 may analyze the data received from the control and measurement unit 110 and may generate reports and alerts to the mobile devices 160 associated with one or more persons in a site where the urine collection basin 190 and the sensors 103-105 are located. As described below, the electronic devices 150 may use machine learning to track health condition of one or more persons using the urine collection basin, and to perform disease prediction. In other embodiments, the processor(s) 120 may analyze the sensors' 103-105 data and may generate reports and alerts to the mobile devices 160.

With further reference to FIG. 1, the computer readable media 125, may be volatile memory and/or non-volatile memory, to store data and/or computer readable instructions that are needed by the processor(s) 120 to communicate with the potentiostat(s) 115, the client devices 160, and/or the electronic devices 150.

The power source 135, in some embodiments, may be one or more batteries, which may be rechargeable and/or replaceable. The power source 135, in some embodiments, may be a power adaptor that may receive power from an electric outlet (not shown). The power source 135 may provide power to the components of the control and measurement unit 110 and/or the sensors 103-105 through one or more wires 185 (shown by dashed lines). Some embodiments may include an on/off switch. The on/off switch may be used to turn off the power from the power source 135 to stop data collection and reporting. The wireless transceiver(s) 130 may include one or more of Wi-Fi, Bluetooth, Zigbee, etc.

The sensors 103-105 installed inside a urine collection basin 190, in some embodiments, may measure electrochemical properties of several persons' urine. For example, the urine collection basin may be a toilet in a household, a care facility, a hospital, etc., where multiple persons may use the same toilet. In these embodiments, the health monitoring urinalysis system 100 may identify each individual person who is using the same urine collection basin, and may use the measurements of each person's urine for tracking the health condition and performing disease prediction of that person. The processor(s) 120, in some embodiments, may use the onset of temperature change to automatically trigger a test.

A person who may want to be identified by the health monitoring urinalysis system 100 (e.g., by an identification such as, a name, a nickname, a username, etc.) may register with the system 100. For example, the person, or an authorized caretaker, may provide the person's identification, health issues, etc., to the system 100. One or more different methods of providing the person's identification may then be used when the person uses the urine collection basin 190.

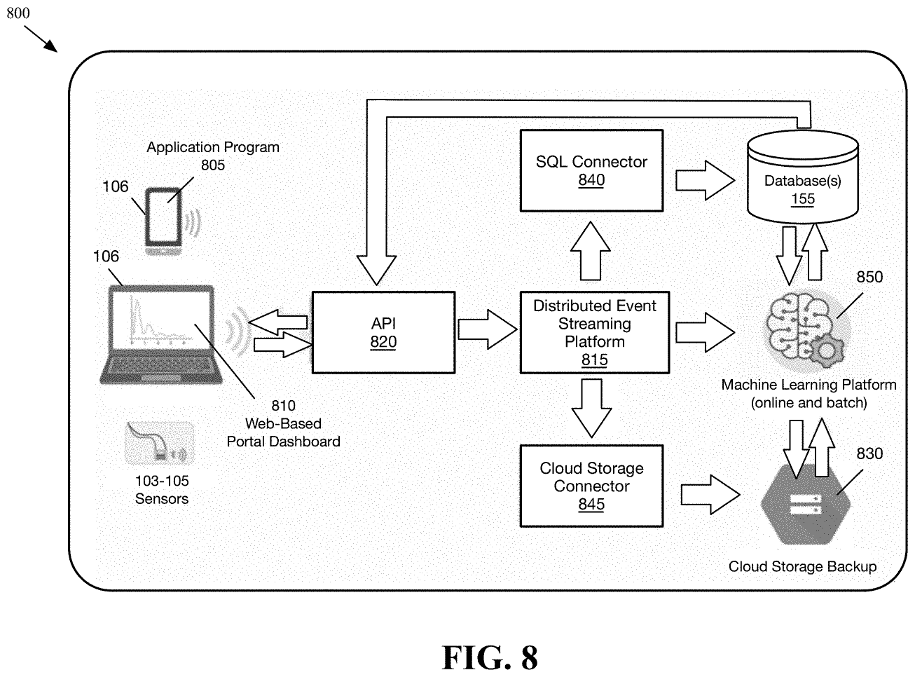

For example, the person may carry a mobile device 160, such as a smartwatch or a smartphone. When the person is going to use the urine collection basin 190, the mobile device 160 may provide the identity of the person to the processor(s) 120. For example, the mobile device 160 may include an application program 805 (FIG. 8) that is associated with the system 100. The application program may be set up to provide the identity of the person through the wireless transceiver(s) 130 to the processor(s) 120. When the urine collection basin 190 is used and the mobile device 106 of the person is within a threshold distance of the urine collection basin 190 (e.g., as determined by mobile device's signal strength), the processor(s) 120 may attribute the sensor 105 readings to that person.

A person, in some embodiments, may provide the person's identification by voice. The person may have provided the person's identification to the health monitoring urinalysis system 100 when the person have registered to use the system 100. The person may then provide the person's identification by voice when the person is using the urine collection basin 190. The control and measuring unit 100, in these embodiments, may include a microphone 140 that may capture voice. The processor(s) 120 may receive the voice captured by the microphone 140 and may perform voice analysis to identify the person's identification.

Some embodiments may install a fingerprint reader 145 close to a urine collection basin 190. The fingerprint reader may be used by a person to provide the person's identity. The person may have provided the person's identification and an image of the person's fingerprint to the system 100 when the person have registered to use the system 100. The person may then provide the person's identification by touching the fingerprint reader 145 when the person is using the urine collection basin 190. The processor(s) 120 may use the fingerprint reader's reading of the person to identify the person.

The health monitoring urinalysis system 100, in some embodiments, may use machine learning to identify a person from the electrochemical responses of the person's urine after several times that the person has provided the person's identity when using the urine collection basin 190. As described below, the machine learning may provide several technical advantages, including, but not limited to, identifying the persons who want their urine analysis data to be collected. These persons may be identified from the characteristics of their urine and may not be required to identify themselves after each use of the urine collection basin.

If a person does not provide identification, the health monitoring urinalysis system 100 may be configured to either record the data anonymously or to ignore the data when the data cannot be related to a known person. For example, if the sensors 103-105 are installed in a bathroom of a house, the system 100 may be configured to ignore the measurements associated with guests, or the measurements associated with any resident who does not want to be identified to avoid gathering results from unknown subjects to respect their privacy.

As another example, if the sensors 103-105 are installed in a bathroom of a cancer research center, there may be a good chance that a large number of the patients may have cancer. The persons using the bathroom may consent to their data to be collected. Collecting data from a large number of persons may provide valuable data for identifying markers of different diseases and health conditions in the urine.

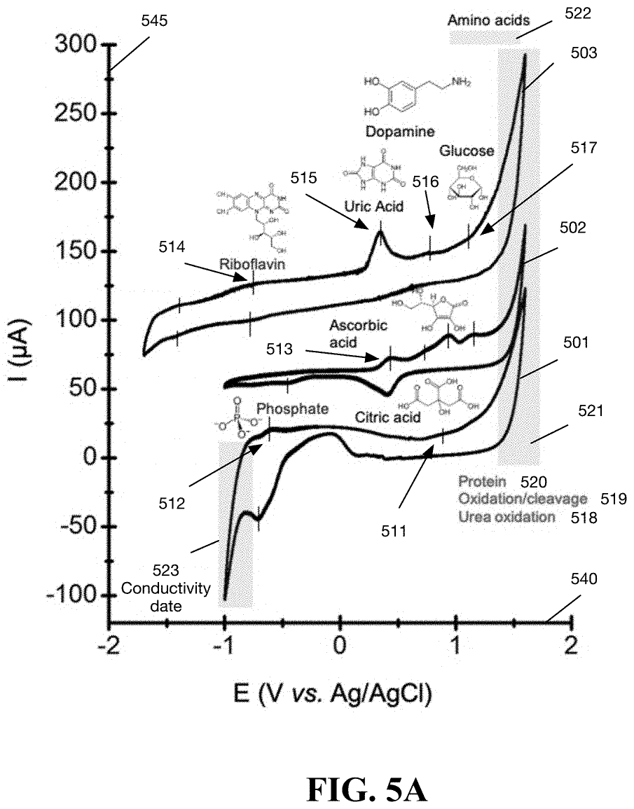

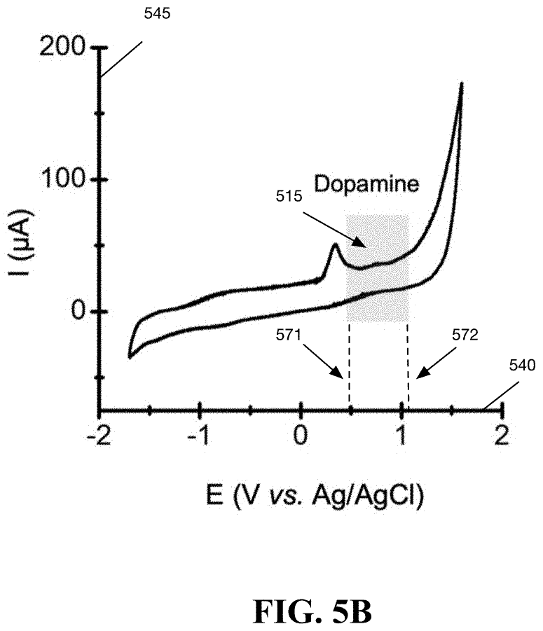

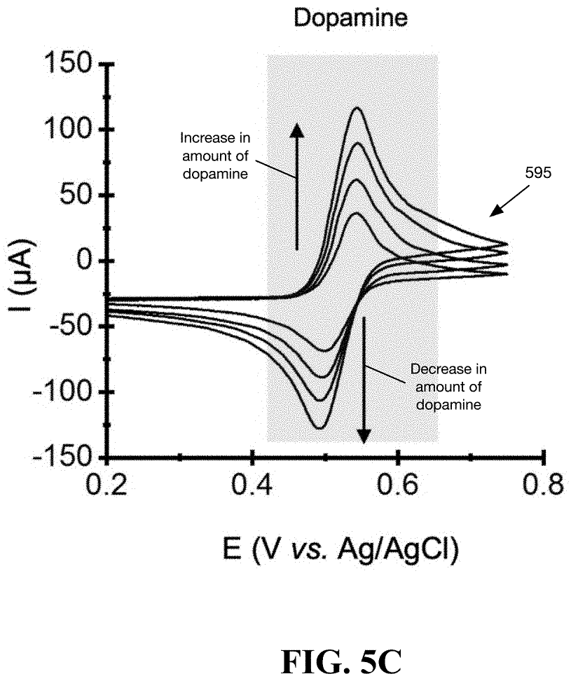

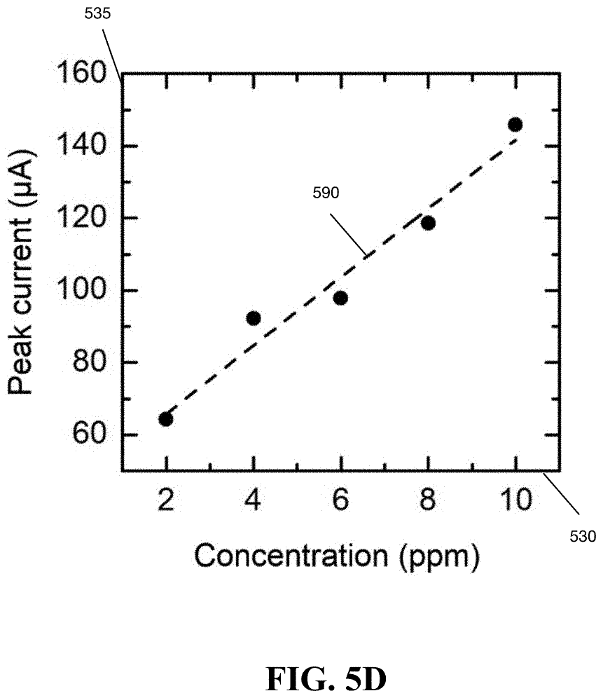

FIG. 5A illustrates an example of the responses of three electrochemical sensors from the analysis of a midday urine, according to various aspects of the present disclosure. The graphs 501-503 may be drawn as graphs that show the voltage applied between two of each sensor's electrodes on the horizontal axis 540 and the resulting current on the vertical axis 545.

In the example of FIG. 5A, the graph 501 shows the response of a sensor with platinum electrodes, the graph 502 shows the response 502 of a sensor with gold electrodes, and the graph 503 shows the response 503 of a sensor with glassy carbon electrodes. Each sensor's response may include several voltage and current pairs. The measurements of the temperature sensor 103 and the pH sensor 104 are not shown in FIG. 5A.

The electrochemical response of each electrode may include one or more markers 511-517 that may indicate the presence and the mount of different metabolites present in the urine. As shown in the example of FIG. 5A, the system 100 (FIG. 1) may report on features related to uric acid 515, dopamine 516, phosphate 512, citric acid 511, vitamins (ascorbic acid 513 and riboflavin 514), and glucose 517. The system 100 may report on features related to, urea oxidation 518, oxidation/cleavage 519, and proteins 520 (which are in the same range of voltage 521). The system 100 may report on features related to amino acids 522 (identified by the shaded range of voltage). The features may relate to analytes and/or metabolites. A metabolite is a substance that is an intermediate or end product of metabolism. An analyte may be any substance of interest in a liquid, such as, for example, and without limitations, a metabolite or an impurity. The measured metabolites are known as biomarkers for many human conditions and habits. For example, urinary proteome analysis for early diagnosis and detection of heart problems, coronary, and artery diseases. Some embodiments may include a sensor for an electrochemical human chorionic gonadotropin (hCG) test, which identifies pregnancy as well as other health conditions.

With further reference to FIG. 5A, the response of the sensor with platinum electrodes (the graph 501) may include conductivity data 523 of urine. In the lower voltage range (e.g., around −1 volts), the sensor with platinum electrodes may split the water molecules (e.g., to O2 and H2). Based on what ions are in urine, the amount of H2 differs. The conductivity data 523 shows the total number of ions in urine. Ionic conductivity is a general data which may be related to many health conditions in clinical studies.

FIG. 5B identifies the portion 570 of the response 503 of the sensor with glassy carbon electrodes of FIG. 5A that relates to dopamine, according to various aspects of the present disclosure. As shown in FIG. 5B, the dopamine response is received in response to applying a voltage between approximately 0.5 volts 571 and 1.05 volts 572.

FIG. 5C illustrates graphs 595 showing the response from a set of dopamine reference solutions with increasing concentration, according to various aspects of the present disclosure. The sample used to in FIG. 5C is generated the lab with water, dopamine, and other substances to make the sample the same as the base of urine. The amount of dopamine is then changed in order to generate each individual graph.

The figure shows the calibration curve of maximum positive current observed (peak current) vs. concentration of dopamine using the carbon electrode sensor which is used for quantitative determination of dopamine content in urine. Same analysis may also used for other compounds monitored by the device.

FIG. 5D illustrates a graph 590 that shows the peak currents 535 of a set of reference solutions at particular voltages 530. The data collected by the sensor in FIG. 5D may be used to quantitatively determine the concentration of analyte of interest (in this example dopamine) by comparing the peak current at a particular voltage measured from urine during a test (e.g., as described above with reference to FIG. 1) with the peak currents of the set of reference solutions which are used to precalibrate the PHMD device 101.

The health monitoring urinalysis system, in some embodiments, controls and records the response from multiple SPE in addition to temperature and pH (e.g., responses from a total of 10 sensors) simultaneously. Example of the materials for electrodes on SPE may include Activated Carbon, Carbon MWCNT, Pt nanoparticles on carbon, Au nanoparticles on carbon, Ag nanoparticles on carbon, NiOx modified Carbon, CuO modified Carbon, reduced graphene oxide carbon.

The results shown in FIGS. 5A-5D illustrate the methodology in using knowledge-based analysis of sensor readings to quantify selected metabolites and to use machine learning (regression models) and pre-quantified references for quantification, as well as developing associations between the target responses and particular conditions. As described below, the system of the present embodiments may use machine learning for real time quantification of large numbers of target metabolites. A significant consequence of collecting urine data multiple times a day and the resulting large datasets is determining the normal levels of known and unknown features as machine learning models are trained on more data points.

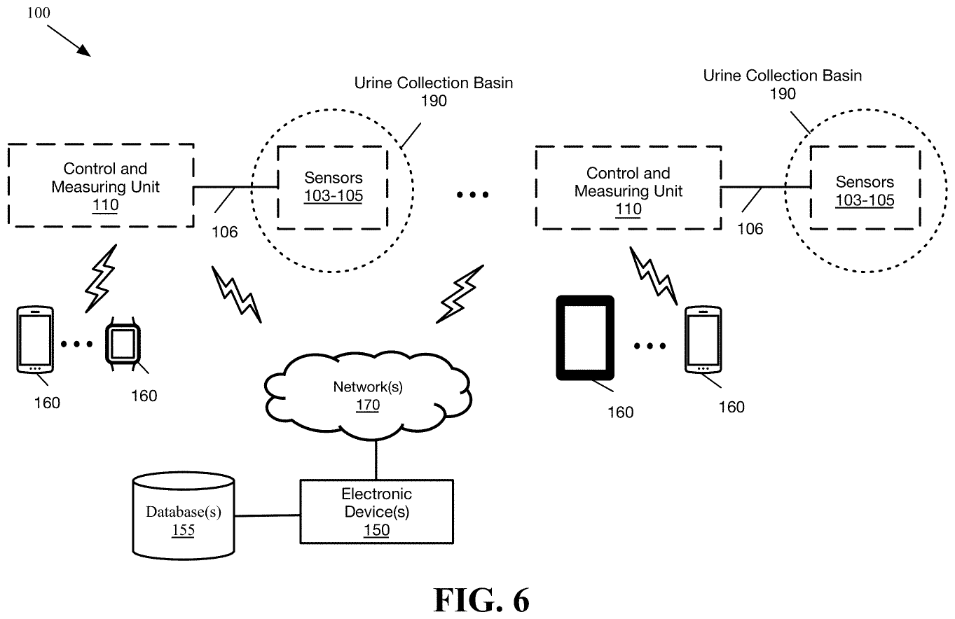

The health monitoring urinalysis system 100 may include several groups of sensors 103-105 that are installed inside different urine collection basins at different locations. FIG. 6 is a functional block diagram illustrating an example health monitoring urinalysis system 100 with several groups of sensors installed inside different urine collection basins at different locations and the corresponding control and management units, according to various aspects of the present disclosure.

With reference to FIG. 6, the health monitoring urinalysis system 100 may include several groups of sensors 103-105. Each group of sensors 103-105 may be installed inside a different urine collection basin 109. Each group of sensors 103-105 may be connected to (e.g., by a set of wires 106), and may be controlled by, a corresponding control and management unit 110.

The control and management units 110 may wirelessly communicate with one or more client devices 160. The control and management units 110 may communicate with the electronic device(s) 150 through one or more network(s) 150, such as the Internet, intranets, cellular networks, etc. Each group of sensors 103-105 and the associated control and management unit 110 may, therefore, function as an Internet of Things (IoT) device.

With reference to FIGS. 1 and 6, the sensors 103-105 and the control and management unit 110 may be used for different applications, such as personal use, healthcare providers use, research use, etc. For example, individuals or persons living in a house may install the sensors 103-105 in their own toilet for recurrent monitoring of urine. Using the system of present embodiments provides the technical advantage of reducing the inconvenience of regular laboratory visits, especially for elderly population. In addition to a routine urine test, the system of the present embodiments provides constant health monitoring and diagnosis, particularly crucial for high-risk individuals. Furthermore, the system may be able to diagnose diseases and predict health trends.

Healthcare providers, such as hospitals and health clinics, which perform regular urine testing, may use the system of the present embodiments to facilitate the process of testing, and decreasing the chances of getting contaminated samples. Hospitals and clinics may install the sensors 103-105 and a corresponding control and management unit 110 in patients' rooms to perform frequent and hassle-free urine testing. Furthermore, the health monitoring urinalysis system 100 of the present embodiments may be used to collect large-scale data over time for many individuals to monitor general health, diagnose diseases, and predict health trends.

Researchers in biotechnology, in pharmaceutical companies, universities, etc., may use the health monitoring urinalysis system 100 of the present embodiments to research targeted treatment development. The health monitoring urinalysis system 100 may provide scientists and researchers access to anonymized database(s) 155. Some embodiments may provide sample datasets to the universities.

The health monitoring urinalysis system 100 of the present embodiments allows frequent and accessible health monitoring. The individuals may be provided with hassle-free, frequent, accessible and contamination free urine tests in the comfort of their homes or in a hospital/health clinic. The health monitoring urinalysis system 100 is easy and convenient for any individual. Through easy setup of the device in a toilet, the individuals may be able to monitor their health using an application program that may run on a client device 160 and/or may access their data through the networks 170 (e.g., the Internet).

The health monitoring urinalysis system 100 of the present embodiments may provide early diagnosis and may predict health trends and may provide risks associated with diseases to individuals based on the metrics in their urine samples. Access to the classified large-scale data collected from consenting persons may be provided to researchers and scientists for the purpose of research and development of new treatments based on the association between the health metrics and conditions. The data is anonymized to eliminate any security and privacy issues involved with the collected data. The data may be end-to-end encrypted from the control and management units 110 to the database(s) 155 in server storage.

A. Application Program, Online Portal, and Data Collection Infrastructure

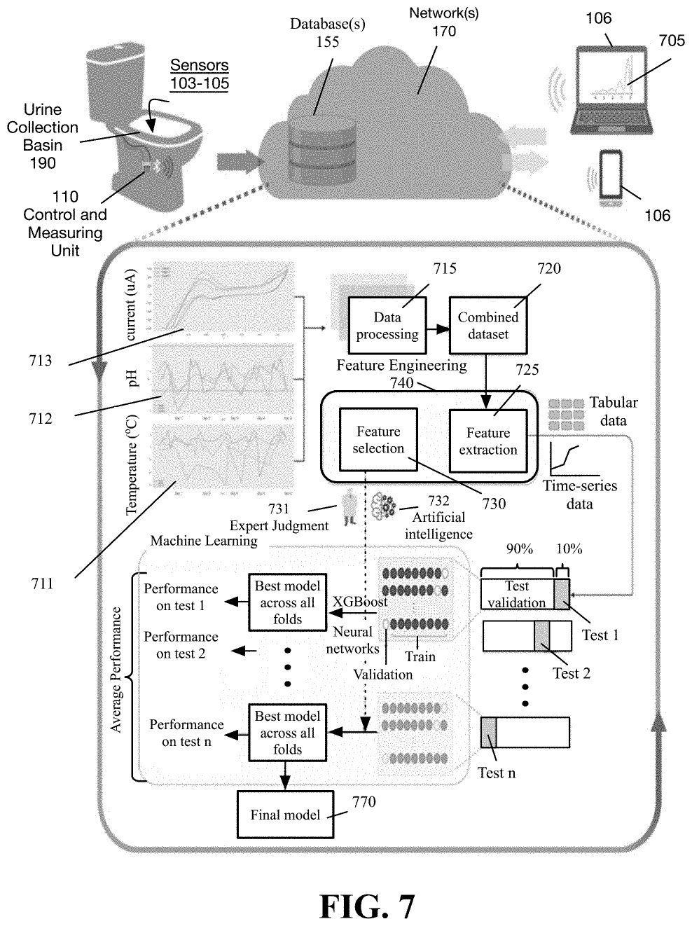

FIG. 7 is a functional diagram illustrating an example platform for recurrent urinalysis and developing a machine learning model to infer health insights, according to various aspects of the present disclosure. With reference to FIG. 7, in personalized setups, such as home use, each individual may be identified through a paired device 160 or other methods, such as voice, fingerprint, etc., as described above. The individuals may access personal data 705 in real time using an application program 805 (FIG. 8) and/or a web-based portal dashboard 810 (FIG. 8) that may be accessed through the network(s) 170. The web based dashboard may be provided on a portal for the users to track personal health metrics, historical trends, and receive health alerts and recommendations. Each individual person may provide personal attributes and health conditions. Data from consenting persons may be used to develop a more precise machine learning model, which may provide more accurate health insights for all users.

In clinical setups with maximum security, the machine learning model may process data locally (e.g., by the processor(s) 120 of FIG. 1) using on-chip inference. Even though the health monitoring urinalysis system of the present embodiments provides personalized health data of patients to the patients and authorized health care providers, the data used for the machine learning model is anonymized and personally identifiable information (PII) is redacted.

With reference to FIG. 7, data may be collected by the sensors 103-105 and may be transferred, for example, in real time, by the control and management unit 110. The raw data, such as temperature measurements 711, pH measurements 712, and data 713 from the sensors 105 may be preprocessed (as shown by 715) and may be combined (as shown by 720) before extraction (as shown by 725) and selection (as shown by 730) of machine learning features. The two main approaches for conducting feature selection 730 are expert judgment (i.e. knowledge-driven) 731 and automatic feature selection (i.e., data driven) 732, which depending on the context, any or both of them may be applied. The data may go through a multi folded cross-validation before the final model 770 is trained for real time prediction. Machine learning models may improve as more data is collected.

Constant extraction of data from the sensors 103-105 across many individual urine collection basins may yield a very large dataset. For example, a week of raw sensor readings for an individual urine collection basin may exceed 1 MB of uncompressed data. This volume of data may lead to 1 GB of data for twenty individual urine collection basins a year, which immediately suggests that even data from a few thousand urine collection basins may require a large data infrastructure. FIG. 8 is a functional diagram illustrating an example data infrastructure used for handling data flow, according to various aspects of the present disclosure.

With reference to FIG. 8, the scalable data collection infrastructure 800, in some embodiments, may be based on the streaming platforms 815. The streaming platforms 815 may provide a distributed, unified platform for handling real-time data feeds from different sources. The sensors' 103-105 readings data may be sent (e.g., by the control and management units 110 of FIG. 6 and/or the client devices 160) through the APIs 820 and may be ingested by the streaming platforms 815. The data gets persisted on the database(s) 155 and the cloud storage 830 as backup. The data may be stored from the distributed event streaming platform 815 into the database(s) 155 through a structured query language (SQL) connector 840. The data may be stored from the distributed event streaming platform 815 into the cloud storage backup 830 through a cloud storage connector 845. This data may then be used by the machine learning platform 850 for training models and by the application program 805 and the web-based portal dashboard 810 for reporting.

The application program 805 may provide a personalized platform for each registered person and may control sharing of information with others based on the person's preferences. The application program 805 may lock after a time period from the last access, and may be activated by face recognition, voice recognition, fingerprint, password, etc. The registered persons may refuse to collect data or may delete data through the application program 805.

It should be noted that the sensors 103-105 and the control and management units 110 of the present embodiments may be used differently in different embodiments. In the embodiments depicted in FIGS. 1 and 6, the sensors are installed in a urine collection basin 190 and data collection starts automatically once the urine collection basin 190 is used by monitoring changes in the temperature and pH of the liquid inside the urine collection basin 190.

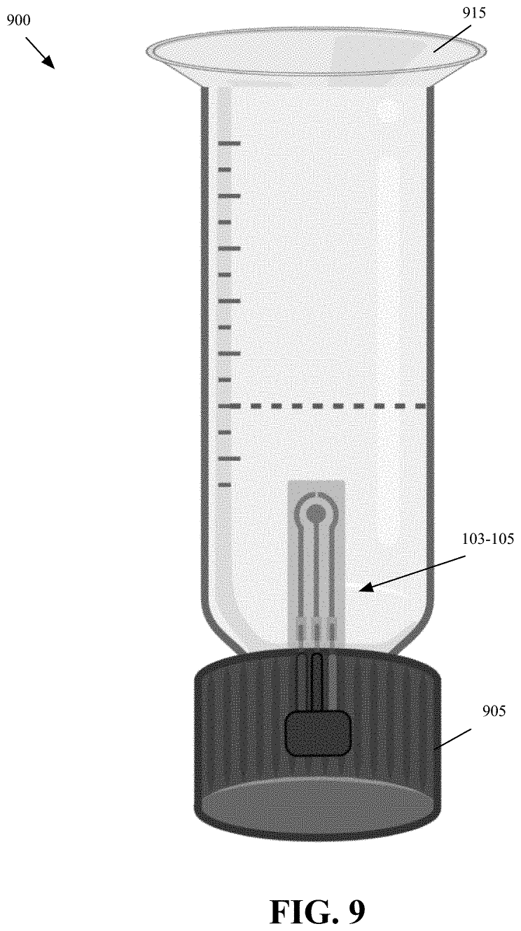

In other embodiments, data collection may be activated by the application program 805. In some embodiments, the sensors 103-105 may not be installed in a urine collection basin 190 and urine sample may be applied directly, or through a sample container to the sensors. FIG. 9 is a front perspective of a sample container that includes a group of sensors, according to various aspects of the present disclosure. With reference to FIG. 9, the sample container 900 may be a portable device. The sample container 900 may include a cap 905, the sensors 103-105, and an opening 915. The urine sample may be applied to the sensors 103-105 through the opening 915. In different embodiments, the urine sample may be applied to the sensors 103-105 with or without an amount of water.

The sensors 103-105 may be installed, for example, and without limitations, by fixing the sensors 103-105 to the cap 905. The sensors 103-105 may be connected to the control and measurement unit 110 (FIG. 1) through a set of wires (not shown) that may go through the cap 905. The wires may go through a hole in the cap and the area around the wires may be sealed to prevent the contents of the sample container 900 from leaking out. The cap 905, in some embodiments, may be changed to include different types of sensors 103-105, depending on the application.

Yet in some embodiments, any human body fluid, including urine, saliva, blood, sweat, tear, and plasma may be tested by the system 100. The body fluid, for example, may be applied using a dropcast device and the test may be started using the application program 805. Dropcast refers to a process to cast or place drops of a liquid sample on a solid surface. In these embodiments, the sensors 103-105 may be selected according to the type of body fluid and the type of labels (labels include the identify of a person using the urine collection basin and/or the identify of one or more conditions that are going to be predicted and/or tracked).

The health monitoring urinalysis system 100 may collect data from the sensors 103-105 (e.g., the temperature sensor, the pH sensor, and the electrochemical sensors), through the application program 805, and/or through controlled groups/controlled lab settings. During setup, the sensors may be calibrated using a set of reference solutions. Data may be collected from all sensors 103-105 installed in a urine collection basin 190 when data collection is activated. The sensor measurements may include measurement of the concentration of a set of analytes, such as, for example, and without limitations, calcium, sodium, potassium, ammonia, uric acid, vitamin B2, vitamin B12, vitamin C, ascorbic acid, dopamine, citrate, glucose, phosphate, nitrate, proteins, etc. The health monitoring urinalysis system 100 may provide quantitative measurement of the analytes using preprogrammed calibration and electrochemical analysis. The health monitoring urinalysis system 100 may use machine learning to recognize signal from noise and extract quantitative data. The health monitoring urinalysis system 100 may use machine learning to learn individual habits and health trends.

The health monitoring urinalysis system 100 may collect data through the application program 805, which may run on mobile devices 106 such as smartphones, tablets, and smartwatches. The set up information may include, for example, and without limitations, gender, date of birth, weight, height, specific diseases, and/or specific health conditions for each registered person. The application programs 805 may collect real time information, such as, for example, and without limitations, date, time, and/or location. The application programs 805 may connect to other health applications (e.g., Clue, Fitness, Apple health, etc.).

The application program 805 may receive data form individuals regarding specific health conditions (pain and sting, infection, pregnancy, menstruation, etc.), specific food/drug consumptions, exercise, sleep hours, measurements (e.g., urine, feces, blood, saliva, water, etc.), measuring subject (urine, feces, blood, saliva, water), etc.

The health monitoring urinalysis system 100 may collect data from controlled groups using controlled lab settings. Data, such as, for example, and without limitations, disease and/or health conditions (cancer, diabetes, infections, depression, stress, pregnancy, menstruation. dehydration etc.), ethnicity, gender, age groups, specific food and/or drug consumptions, known subjects (urine, feces, blood, saliva etc.) may be collected from the controlled groups.

B. Machine Learning Platform

Some embodiments may use machine learning to extract metabolic data, to provide personalized health insights, to identify irregularities related to human conditions, and/or to provide early diagnosis based on electrical and electrochemical responses of urine received from the customizable group of sensors 103-105. Since human body fluid is ionically charged (ionic conductivities of 1-200 mS cm⁻¹), direct electrochemical measurement with no sample pretreatment, separation, and preparation may be carried out. This natural advantage enables the present embodiments to use measurements made by electrochemical sensors to make a direct analysis in the voltage range of water stability on electrodes (approximately ca. ±1.5 V) versus RHE (reversible hydrogen electrode). Considering this technical advantage, many biomarkers may be extracted from the monitoring of urine samples. Temperature, pH, conductivity, rate of water decomposition, which are known as physiological signals, may then easily be available.

The electrochemical analysis of complex mixtures on well-defined and designed electrodes serves as established or promising clinical methods for determination of biomarkers. Some embodiments may use machine learning to classify the electrochemical response of a plurality of sensors 103-105 (FIGS. 1 and 6), which are placed in the urine, across many individuals. The electrochemical responses of many sensors 103-105 may contain unique characteristics that may be used for accurate diagnosis. For instance, an aqueous solution of acetic acid on the gold electrodes may provide a fingerprint type response different from other analytes on gold and even on different electrodes (e.g., platinum or glassy carbon surfaces). Hence, valuable insights may be available in each electrochemical measurement of urine and intelligent analysis may provide many metrics, which are of interest to monitor health conditions, daily habits, and drug use. Machine learning, in some embodiments, may achieve an accuracy of greater than 90 percent when dealing with complex datasets even against high background noise levels.

The continuous health monitoring and metabolite analysis of human urine using collected data from a multitude of sensors 103-105 (e.g., FIG. 6) of the present embodiments may discover hidden patterns and meaningful relationships leading to identifying human health conditions, behaviors, and disease markers that may be used for diagnosis, preventive health alerts, and developing new personalized treatments. Although measuring the output of each individual sensor, for example, uric acid, pH, and temperature may provide useful information about the health condition and lifestyle, but the output of the group of sensors 103-105 provides complex and low-level datasets. Therefore, mathematical models may be required to transform the raw data from the sensors 103-105 to extract more complex correlations (health conditions) and new insights (unknown biomarkers). Machine learning and deep learning techniques may be used, in some embodiments, in analyzing and extracting correlations from big datasets and complex metabolic information. Trained models on historical data may identify any anomaly and predict future trends in the urine data.

Some embodiments may develop models for complex multiclass multivariable machine learning problems. The output of each individual sensor and the group of sensors 103-105 at low iterations may not provide any decipherable information linking different labels to data trends. However large datasets from sensors 103-105 (FIG. 6) installed at multiple locations may be used by the machine learning models of the present embodiments to provide a statistically significant correlation.

With further reference to FIG. 7, following steps may be used in some embodiments to perform machine learning on the acquired data. For feature engineering 740, the low-level sensor datasets may be probed for suitable attributes to be used in the machine learning algorithms, which may particularly be important in dealing with the measurement of noise and missing pieces in complex datasets. Some embodiments may apply smoothing algorithms to low-level sensor datasets to remove noise. Additionally, depending on the situation, some embodiments may apply different chemical compounds to lower the possible noises in the measurements.

In order to identify appropriate features for improving the quality of developed machine learning models, some embodiments may use one or more of the following strategies. The feature extraction 725 may include a series of different transformations to translate the raw data into the required high-level input. The features may be the voltage and current pair values measured by a sensor within a particular range of voltages. For example, as shown in FIG. 5B, the feature dopamine may be associated with the voltage and current value pairs that the sensor with glassy carbon electrodes measures in the voltage range from approximately 0.5 volts (as shown by 571) to approximately 1.05 volts (as shown by 572). Other features may be associated with any of the analytes and metabolite 511-523 measured by different sensors as shown in FIG. 5A.

It should be noted that the embodiments that use machine learning with automatic feature selection may identify features (e.g., local maximum or minimums in current values measured by a particular sensor within a range of voltage) for a particular label (e.g., for patient with a known disease, persons with a known diet, persons with exposure to a known hazardous material) without correlating the features to a particular metabolite or analyte. For example, a majority of persons with exposure to a particular hazardous material may be showing a peak in the current value measurements for the voltage range of 1 to 1.5 volts made by a sensor with platinum electrode. The system 100 may then generate an alert indicating a possible exposure to the particular hazardous material when the peak in the current values in that voltage range of 1 to 1.5 volts measured by a sensor with platinum electrode exceeds a threshold.

Different embodiments may use one of the two strategies for feature extraction in developing the machine learning models. The first strategy is the “current intensity,” which uses the current amplitudes at each voltage (or potential) in electrochemistry for analysis. The current amplitudes may be used as features for the machine learning model. The input features may be defined as:

Xᵥ=(I₁, I₂, . . . , I_{N}), where Iₑ is the current intensity of voltage v of experiment e∈{1, 2, . . . , N}, v is the applied voltage in the probed range, for example, −1.8V to 1.8V, and N is the number of experiments. For each sensor in the sensor group 103-105, an Xᵥ column in a matrix is defined. In this strategy, Machine Learning algorithms such as support-vector machines (SVM), decision trees, and boosting algorithms may be used.

The second strategy for defining input data is sequence modeling. This approach for defining input data includes treating the readings of each sensor as a time-series vector where each sensor is a feature for the machine learning model. This problem may be formulated with T observations as Xᵢ=(I₁, . . . , I_{T}) (the time series). The input data may then be a matrix X=(X₁, . . . , X_{N}) for N data measurements from sensors. In this strategy, Deep Learning algorithms such as Neural Networks may be used.

Feature selection 730 refers to selecting important features and reducing dimensionality of a complex dataset and is required in many bioinformatics applications due to complex nature of datasets. Since the amount of data extracted from the sensors 103-105 may have high dimensionality and not all of the sensor readings are relevant to a target label, feature selection 730 may employed.

Some features and chemical components available in the urine are already known to be associated with specific health conditions. For example, glucose is not normally found in urine. However, the extra glucose in the blood is eliminated through the urine. Therefore, glucose may be targeted as a screening test for diabetes. The feature selection 730 may be used to quantify the amounts of target metabolites and make associations between data features, events, conditions, and diseases.

Such associations may replace the traditional urine tests by measuring the target features and may be used in research and development of pharmacometabolomics. Quantification of target metabolites may be performed through knowledge-driven selection of the features. Data driven feature selection may enable association to traits observed in specific conditions. For example, people with kidney problems may show particular traits that may be used in early diagnosis for high risk individuals. For more complex diseases such as cancer, automatic feature selection may replace or complement the expert judgment feature selection by finding new patterns and correlations in the large amount of data. Constant monitoring of selected features, provided by the system of the present embodiments may, therefore, enable personalized health recommendations and diagnosis.

For machine learning model development, some embodiments may implement multiple machine learning models targeted toward different objectives. For current value features, some embodiments may use XGBoost (eXtreme Gradient Boosting), which is a machine-learning method that combines weak learners (decision trees) to achieve strong class prediction. In XGBoost, a series of decision trees classify labelled data that is used for the training of the model. Each decision tree is comprised of many branches, which iteratively split the training data semi-optimally to reduce errors and improve the accuracy of the resulting classification. For sequence modeling approach, some embodiments use Deep Convolutional Neural Networks (CNN), Recurrent Neural Networks (RNN), transformers and attention models. A relevant example, which may be used in some embodiments, is InceptionTime algorithm, as it exceeds the accuracy and speed of previous sequence classification algorithms. In both cases, the probability of the assigned labels {y¹, . . . , y^{k}} (a particular condition) is calculated by the classification model once it is trained on a set of data.

In addition to constant monitoring of urine temperature (which directly correlates to the body temperature) and pH, the present embodiments use targeted electrochemical measurements to report on a set of urine metabolites and use machine learning to provide detailed health trends and insights. One of the advantages of the present embodiments is, in addition to a routine urine test, providing constant health monitoring that may diagnose abnormal conditions at early stages. Furthermore, unlike single use devices for testing ovulation, pregnancy, urinary tract infection, and specific diets (e.g., keto diet), the system of the present embodiments provides such metrics in real time (with no direct human intervention), collects historical health data, analyzes information, monitors trends continuously and provides health insights.

The machine learning empowered interpretation of large-scale urinalysis over time for many individuals provides the capability to collect urine metabolome as a fingerprint of health conditions, to monitor health, predict trends and diagnose diseases by finding the association between various health metrics and conditions. Therefore, the health monitoring urinalysis system 100 of the present embodiments is a platform for capturing health insights and disease prediction. This aspect has the potential to be transformative in the healthcare industry as many diseases are not diagnosed until the critical stages, when the best and the most cost-effective interventions are not compelling. Having a preventive health platform that detects disease symptoms early on by using automatic urine tests is innovative and original.

One of the technical advantages of the health insights provided by the health monitoring urinalysis system 100 of the present embodiments over a typical urinalysis is that the large-scale data collected by the system 100 may enable early detection of many chronic diseases such as cancer, heart diseases, diabetes, kidney diseases, neurological diseases, obesity, and mental disorders, among others. The system 100 may be able to predict conditions causing fever and changes in urine pH, for example, viral and bacterial infections. Constant monitoring of health metrics of many individuals may provide classified data linking a set of responses to specific behaviors, conditions, diseases, and even onset and progression of epidemic and pandemics. Such attributes are possible with the large and frequent sets of classified data that are collected from humans' urine specimen. The system 100 of the present embodiments may also serve as a single device that is able to track lifestyle factors such as diet, drug consumption, exercise, and sleep, among others. Therefore, the system 100 may replace many commercial single use devices for such purposes. The application program 805 and the web-based portal dashboard 810 may enable the users to see a complete report of their health metrics on their smart devices. The report may include health scores and alerts, and therefore, may be made to be easily understandable.

To develop machine learning model (supervised/unsupervised), some embodiments may define features and labels as sensor readings (voltages/current, pH, temperatures, etc.) and collected data (time, gender, health conditions, etc.). The data may be split to train validation and test sets using cross-validation. The model, in some embodiments, may be trained on the train-set and hyperparameters may be selected on the validation-sets. The performance of each model may be reported on blind test-set. The best machine learning algorithms may be selected based on the performance results (Supervised: accuracy, precision, recall, f-score, AUC; Regression: R square, mean square error, mean absolute error; Unsupervised: silhouette index, Davies-Bouldin index, Calinski-Harabasz, etc.) depending on the problem definitions. For example, for predicting pregnancy, the aim may be achieving high precision/low false-positive while for some diseases, such as, cancer/Coronavirus Disease 2019 (COVID-19) may be high recall/low false-negative. The models may be updated on a regular basis based on the amount of new data gathered.

Data cleaning and preparation, in some embodiments, may be performed as follows. Sensors' lifespans may be predicted by measuring performance of sensors on water by applying unsupervised machine learning methods for outlier/noise detection. A notification to change the sensors may be sent through the application program 805. Date of changing the sensors may be used as a feature in machine learning models to decrease noises by learning sensors' performance pattern. Machine learning models may also be trained and applied based on different sensor condition categories (such as, new, usable, old, etc.).

The subject may be predicted (in a case that the subject is not specified by the user) as follows. Multiclass multilabel (such as, urine, feces, blood, saliva, water, etc.) models may be trained on the labeled datasets and applied on the new samples which are considered as new features that describe a collected sample. Separate machine learning models may also be trained on each class. Unsupervised machine learning model may be applied to cluster samples and may extract the majority class in each cluster (when there is not have enough labeled samples).

In some embodiments, the machine learning models maybe developed based on two strategies: Known targets/markers and Unknown targets/markers. Known targets/markers may include targeted chemical compounds and/or disease and health conditions. The targeted chemical compounds may be inferred through quantifying target metabolite using preprogrammed calibration of current and voltage responses. A regression model may be trained on labelled samples (known voltage and current) and may be applied on new samples to estimate the amount of these chemical compounds in a sample and predict known specific health conditions related to the measured markers.

Regression models may be used to predict the amount of chemical compounds based on sensor readings. The regression models may take sensor readings and find a mathematical relationship between them in order to predict the amount of different chemical compounds. These chemical compounds include, but not limited to, sodium, calcium, potassium, chloride, phosphate, and/or magnesium. In addition to predicting the exact amount of the compounds, the results also may be classified to low or high on electrolytes.

The disease and health conditions may include, for example, and without limitations, diabetes, pregnancy, menstruation, food or drug consumptions, dehydration, etc. A supervised machine learning model (such as, neural networks, support vector machine, random forecast, XGBoost, etc.) may be developed on samples with labels (labels include the identify of a person using the urine collection basin and/or the identify of one or more conditions that are going to be predicted and/or tracked). The trained model may be applied on new samples to predict each condition. The developed models may be interpreted to extract important markers. For example, a low fruit and vegetable diet may decrease the pH of urine and the urinary excretion of potassium and citrate, while it may increase the amount of calcium in urine. Therefore the supervised model may be trained on the low and high vegetable diet labels as a binary classification, and the model may predict and/or track sample diet conditions. Also, some diseases, such as, urinary tract infection (UTI), are associated with availability of different markers, such as, nitrites, leukocytes, and blood. Therefore a supervised trained machine learning model may predict UTI and by interpreting the model the abnormal value for each marker may be calculated.

A regression model, in some embodiments, may be trained on the training data to predict continuous labels. These labels may be used to track the trends and different conditions. For example glucose in urine is a marker of diabetes or excess vitamin C consumption is excreted from urine. High/low amounts of each compound may be associated with specific health conditions and/or food consumptions, and also for tracking and/or predicting some patterns like menstruation time, period cycle length, cycle duration, ovulation time, etc., in female samples.

Unknown targets and/or markers for predicting and/or tracking new diseases and/or health conditions, such as, cancer, depression and anxiety, etc., may also be used to develop machine learning models in some embodiments. A supervised machine learning model may be developed on collected data (such as, sensor readings, clinical and installation data) as features and a targeted label (such as, cancer, depression and anxiety, etc.). If the targeted phenotype predicted was predictable with high accuracy and a correlation is found between the collected features and the label by interpreting the model, important features may be extracted. For example, the level of current for some voltages measured by sensors may discriminate between normal and cancerous samples with high accuracy. Then, as voltage is a chemical compound fingerprint, these voltages may reveal new markers related to cancer/any targeted label.

The post-processing of data, in some embodiments, may include extracting biological data, providing personalized health insights, detecting irregularities related to human conditions, and providing early diagnosis. The results may be reported, for example, by one or more of the following methods. The application program 805 may include different blocks that the user may activate. Each block, such as, a diet block (ketone, hydration, food/vitamins consumption, etc.), female block (pregnancy, menstruation, ovulation, etc.), health block (disease, infection, mental disorders, etc.) and other customized blocks may report and/or track specific markers and/or health conditions.

For a particular person and a specific task, previously trained machine learning models may be applied to provide recommendations for calculating intensities of each compound using machine learning models trained for the task, food recommendation using machine learning models trained for the task, water recommendation using machine learning models trained for the task, ovulation prediction using machine learning models trained for the task, mood prediction using machine learning models trained for the task, health/disease monitoring using machine learning models trained for the task, etc.

A user may share the results with other users/doctors using the application program 805, email, messages, etc. A user may define different notification settings for specific conditions, such as dehydration, period time, ovulation time, risk of any diseases, etc.

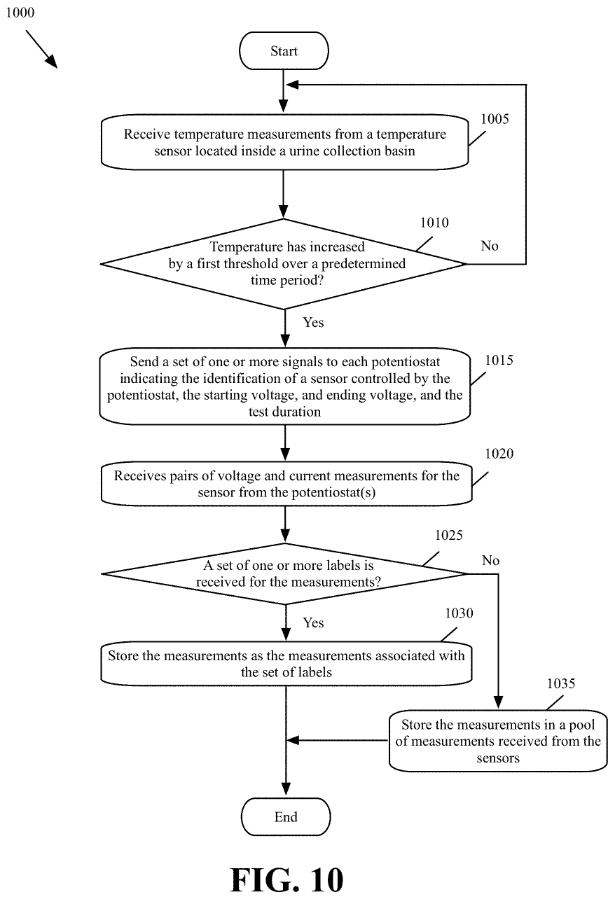

FIG. 10 is a flowchart illustrating an example process 1000 for collecting data by a PHMD 101, according to various aspects of the present disclosure. The process 1000, in some of the present embodiments, may be performed by a processor 120 of the PHMD 101 of FIG. 1.

With reference to FIG. 10, temperature measurements may be received (at block 1005) from a temperature sensor located in a urine collection basin. For example, a processor 120 of FIG. 1 may receive temperature measurements from the temperature sensor 103 that is inside the urine collection basin 190. The output of the temperature sensor 103, in some embodiments, may be connected to an input port of the processor 120 by a wire, for example, as described above with reference to FIGS. 2 and 3. The processor 120 may read the temperature measurements at a time interval (e.g., once every second, once every 5 seconds, etc.).

Next, a determination may be made (at block 1010) whether the temperature has increased by a first threshold over a predetermined time period. The processor 120 may determine whether the temperature of the liquid inside the urine collection basin 190 has increased by m degrees Fahrenheit over n seconds, where m and n are values greater zero. A temperature increase in the urine collection basin 190 may indicate the urine collection basin 190 is being used.

When the temperature has not increased by the first threshold over the predetermined time period, the process 1000 may proceed back to block 1005, which was described above.

Otherwise, a set of one or more signals may be sent (at block 1015) to each potentiostat indicating the identification of a sensor controlled by the potentiostat, the starting voltage, and ending voltage, and the test duration. For example, the processor 120 may start a test by sending signals to the potentiostat(s) 115 to apply a range voltage to the electrodes of the sensors over a time period and read the resulting currents.

The potentiostat(s) 115, in some embodiments, may be preprogrammed to receive the sensor identifications, the starting voltage, the ending voltage, and the time period, apply the starting voltage and make a current measurement, change the voltage by an increment and make another current measurement, and repeat applying voltage and making current measurements until the end voltage has reached. The potentiostat(s) 115, in some embodiments, may save the voltage and current pairs for each sensor and may send the results to the processor 120 in a file. In other embodiments, the potentiostat(s) 115 may send the voltage and current pairs for each sensor to the processor 120 as the measurements are made.

The pairs of voltage and current measurements may be received (at block 1020) for the sensors from the potentiostat(s). For example, as described above, the processor 120 may receive the pairs of voltage and current measurements in a file or as the measurements are made. A determination may be made (at block 1025) whether a set of one or more labels is received for the measurements.

The labels may be the identification of a person who has used the urine collection basin 190, the identification one or more health issues or illnesses that the person who has used the urine collection basin 190 may have, dietary supplements that the person have taken, food items that the person have eaten, etc. The labels may be received by the processor 120 from a client device 160 (e.g., a smartphone, a smartwatch, a tablet, etc.) associated with the person using the urine collection basin 190. The client device may include an application program 805 (FIG. 8) that may communicate with the processor 120 when the client device 160 reaches a vicinity of the processor 120 (e.g., with the threshold distance). The identification of the person may also be received by the processor through a fingerprint reader 145. The identification of the person may also be captured as voice by the microphone 140 and may be sent to the processor 120.

When a set of one or more labels is received for the measurements, the measurements may be stored (at block 1030) as the measurements that are associated with the set of labels. The process 1000 may then end. When a set of one or more labels is not received for the measurements, the measurements may be stored (at block 1035) in a pool of measurements received from the sensors. The process 1000 may then end.

The stored measurements may be analyzed, for example, as described above with reference to FIGS. 5A-5D and 7-8 to identify analytes and markers of interest, to determine health issues, to determine dietary trends, and/or to generate alerts. Machine learning models may be used during the analysis as described above with reference to FIG. 8. The analysis may also be used to further modify or improve the existing model. The analysis may be done by the processor(s) 120 of the PHMD 101 and/or by the processor(s) of the electronic device(s) 150 (e.g., by the processor(s) of one or more remote servers).

C. Example of Collected Data and Machine Learning Driven Analysis

The health monitoring urinalysis system 100 described above has been tested by running for months in a personal home. A representative set of urine sample results are taken over a 1-week period for three individuals, two middle aged men and a middle-aged woman. The 1-week period was chosen randomly, and the three individuals used the system 100 constantly during this period. The sensors used included four wide range electrochemical sensors (amorphous carbon, glassy carbon, platinum and gold) and standalone sensors for pH and temperature. During the representative trial, 79 urine samples were probed for all three individuals. The protocol is used for multiple electrochemical tests (three for this trial) on the sensors during each collection. The system used the onset of pH and temperature change to automatically trigger a test. Each test took less than 2 minutes.

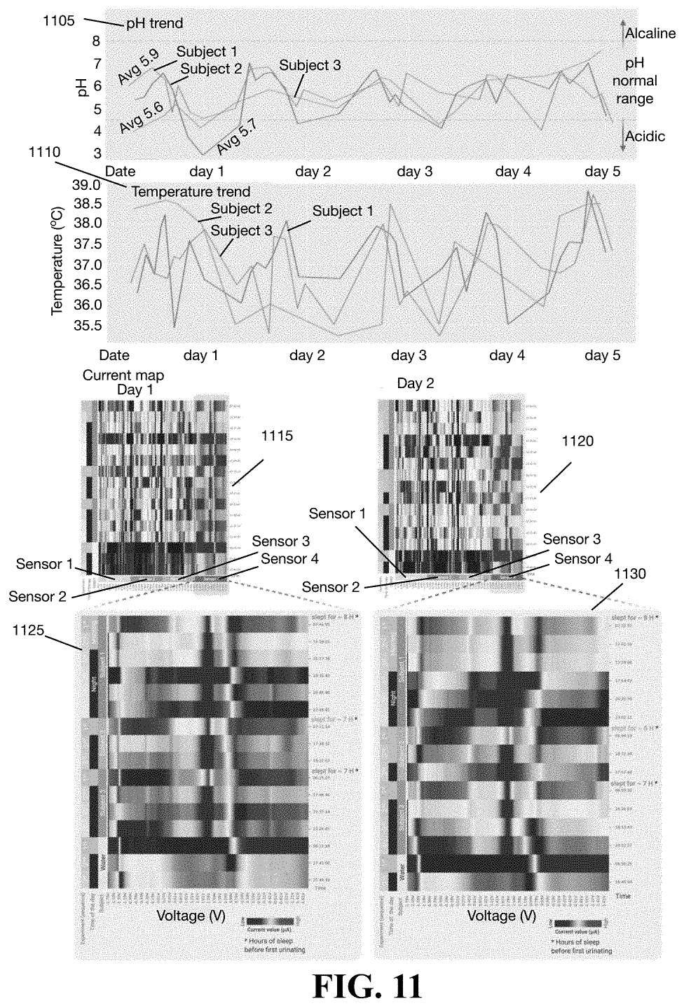

Once the three individuals used the system 100 over the 1-week period of time, the responses from all sensors across all individuals were handled by the system 100, as described above. FIG. 11 illustrates the response from the sensors 103-105 represented as current amplitude maps, pH trends, and temperature trends, according to various aspects of the present disclosure. With reference to FIG. 11, the response from the sensors 103-105 (FIG. 1) are represented as pH trends 1105, and temperature trends 1110, and current amplitude maps of two days 1115-1120.

The current amplitude of two days of all electrochemical sensors at different voltages are indicated. The details of carbon SPE data from two days are shown in the two lower graphs 1125-1130 in FIG. 11. In addition, the graphs show an estimate of the hours of sleep for each individual. The results shown in FIG. 11 illustrate the apparent complexity of the low-level sensor readings. However, as described above, many valuable information may be extracted from such datasets as the present embodiments feed the data into the machine learning models to extract patterns and high-level information. It should be noted that system 100 capability in constant monitoring of temperature and pH of urine may be used in detecting personal lifestyle factors and onset of many health conditions, including fever, urinary tract infections, and other infectious diseases. The system 100 may provide alerts after detecting abnormal health conditions.

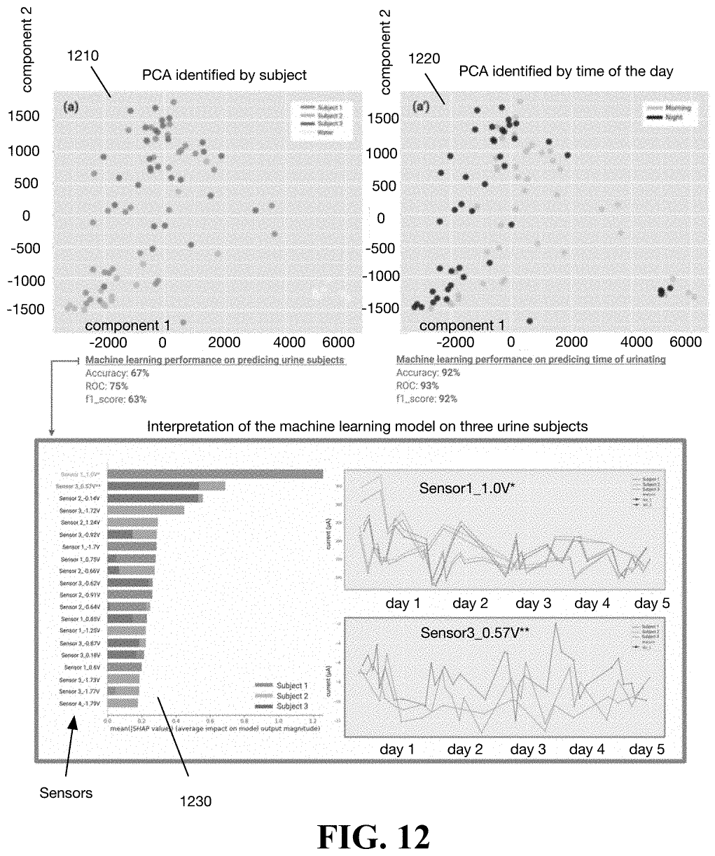

FIG. 12 illustrates example results of XGBoost model, where 80% of the data is used for training, 10% of the data is used for validation and hyperparameter tuning, and 10% of the data is used for unseen test data, according to various aspects of the present disclosure. In addition, the models implemented sequence classification using Tensorflow. The models shown in the example of FIG. 12, have classified “subject” 1210 (three classes of subject1, subject2, and subject3, with baseline of 33% accuracy) and “time of the day” 1220 (two classes of morning and night, with baseline of 50% accuracy) labels with high significant accuracies of 67% and 92%, respectively. Depending on the label chosen for the model, the important voltage(s)/chemical components may be found that play an important role in the subject and time of day predictions. In the example of FIG. 12, the top 20 features, sorted by their average impact for classifying, then grouped by their contribution of each subject are shown.

Principle component analysis (PCA) of high dimensional data of the same two labels shows that a clear pattern is not easily available in lower dimensions. ROC and F1 scores also show high values, indicating the ability of the developed models in learning associations and extracting patterns.

In addition to pH and temperature, the other target metabolites mentioned earlier (e.g., uric acid) may be reported in real time by running the inference of the machine learning models through the application program 805 (FIG. 8) or the web-based portal dashboard 810 (considering the privacy setting of the user) upon each toilet use. The machine learning interpretation of the target metabolites may be associated with certain health conditions. Additionally, important features may be extracted from the trained machine learning models of the present embodiments. Some embodiments use a library (e.g., SHAP—SHapley Additive exPlanations) for explaining the behavior of machine learning models to extract most important features 1230 in classifying the acquired data. Such insights may be used for discovering new metabolites linked to certain conditions, which may be utilized by research institutes for development of personalized metabolic informed treatments.

The present embodiments provide optimization and hyperparameter tuning of the machine learning models and develop novel neural network architectures for classification of sensors' data of all users. The results shown in FIGS. 5A, 11, and 12 illustrate the high capabilities of the system 100 of the present embodiments. As more users with various physiological labels use the system 100, the machine learning platform may make associations to different health conditions, diseases, and personal habits.

In summary, a robust learning model for electrochemical response of urine metabolites and extracting insights from this complex and high noise environment is novel both in approach and resulting scientific advances. This approach enables handling of low-level data from an array of sensors often in high noise and background space. Instant analysis of human urine without pretreatment for quantitative analysis of target analytes, detecting trends, anomaly, and early diagnosis through classification of urine samples across different users is a powerful and novel platform for tracking human health and metabolic fingerprints.

### II. Computer System

Many of the above-described features and applications may be implemented as software processes that are specified as a set of instructions recorded on a computer readable storage medium (also referred to as computer readable medium). When these instructions are executed by one or more processing unit(s) (e.g., one or more processors, cores of processors, or other processing units), they cause the processing unit(s) to perform the actions indicated in the instructions. Examples of computer readable media include, but are not limited to, random access memory (RAM), read-only-memory (ROM), read-only compact discs (CD-ROM), erasable programmable read-only memories (EPROMs), electrically erasable programmable read-only memories (EEPROMs), flash memory (e.g., secured digital (SD) cards, mini-SD cards, micro-SD cards, etc.), magnetic and/or solid state hard drives, ultra-density optical discs, and any other optical or magnetic media. The computer readable media does not include carrier waves and electronic signals passing wirelessly or over wired connections.

In this specification, the term “software” is meant to include firmware residing in read-only memory or applications stored in magnetic storage, which can be read into memory for processing by a processor. Also, in some embodiments, multiple software inventions may be implemented as sub-parts of a larger program while remaining distinct software inventions. In some embodiments, multiple software inventions may also be implemented as separate programs. Finally, any combination of separate programs that together implement a software invention described here is within the scope of the invention. In some embodiments, the software programs, when installed to operate on one or more electronic systems, define one or more specific machine implementations that execute and perform the operations of the software programs.

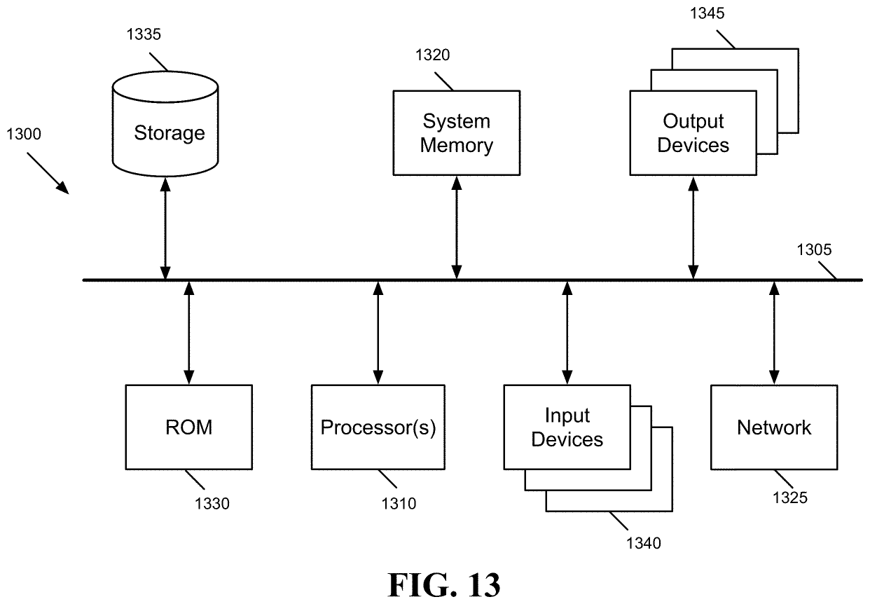

FIG. 13 is a functional block diagram illustrating an example electronic system 1300, according to various aspects of the present disclosure. With reference to FIG. 13, some embodiments of the invention, such as for example, and without limitations, the mobile devices, the electronic devices, control and management units, the servers, etc., described above, may be implemented using the electronic system 1300. The electronic system 1300 may be used to execute any of the processes, methods, controls, or operating system applications described above. The electronic system 1300 may be a computer (e.g., a desktop computer, personal computer, tablet computer, server computer, mainframe, a blade computer etc.), a phone (e.g., a smartphone), a personal digital assistant (PDA), or any other sort of electronic device. Such an electronic system may include various types of computer readable media and interfaces for various other types of computer readable media. The electronic system 1300 may include a bus 1305, processing unit(s) 1310, a system memory 1320, a read-only memory (ROM) 1330, a permanent storage device 1335, input devices 1340, and output devices 1345.

The bus 1305 may collectively represent all system, peripheral, and chipset buses that communicatively connect the numerous internal devices of the electronic system 1300. For example, the bus 1305 may communicatively connect the processing unit(s) 1310 with the read-only memory 1330, the system memory 1320, and the permanent storage device 1335.

From these various memory units, the processing unit(s) 1310 may retrieve instructions to execute and data to process in order to execute the processes of the invention. The processing unit(s) may be a single processor or a multi-core processor in different embodiments.

The read-only-memory 1330 may store static data and instructions that are needed by the processing unit(s) 1310 and other modules of the electronic system. The permanent storage device 1335, on the other hand, may be a read-and-write memory device. This device is a non-volatile memory unit that may store instructions and data even when the electronic system 1300 is off. Some embodiments of the invention may use a mass-storage device (such as a magnetic or optical disk and its corresponding disk drive) as the permanent storage device 1335.

Other embodiments may use a removable storage device (such as a floppy disk, flash drive, etc.) as the permanent storage device. Like the permanent storage device 1335, the system memory 1320 may be a read-and-write memory device. However, unlike storage device 1335, the system memory may be a volatile read-and-write memory, such as random access memory. The system memory may store some of the instructions and data that the processor needs at runtime. In some embodiments, the invention's processes may be stored in the system memory 1320, the permanent storage device 1335, and/or the read-only memory 1330. From these various memory units, the processing unit(s) 1310 may retrieve instructions to execute and data to process in order to execute the processes of some embodiments.

The bus 1305 may also connect to the input and output devices 1340 and 1345. The input devices may enable the user to communicate information and select commands to the electronic system. The input devices 1340 may include alphanumeric keyboards and pointing devices (also called “cursor control devices”). The output devices 1345 may display images generated by the electronic system. The output devices may include printers and display devices, such as cathode ray tubes (CRT) or liquid crystal displays (LCD). Some embodiments may include devices such as a touchscreen that function as both input and output devices.

Finally, as shown in FIG. 13, the bus 1305 may also couple the electronic system 1300 to a network 1325 through a network adapter (not shown). In this manner, the computer may be a part of a network of computers (such as a local area network (“LAN”), a wide area network (“WAN”), an Intranet, or a network of networks, such as the Internet. Any or all components of the electronic system 1300 may be used in conjunction with the invention.

Some embodiments may include electronic components, such as microprocessors, storage and memory that store computer program instructions in a machine-readable or computer-readable medium (alternatively referred to as computer-readable storage media, machine-readable media, or machine-readable storage media) such as the computer readable media 125 of FIG. 1. Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra-density optical discs, any other optical or magnetic media, and floppy disks. The computer-readable media may store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some embodiments may be performed by one or more integrated circuits, such as application specific integrated circuits (ASICs) or field programmable gate arrays (FPGAs). In some embodiments, such integrated circuits may execute instructions that are stored on the circuit itself. Some of the present embodiments may include flexible circuit, also referred to as flexible printed circuit boards (PCBs). The flexible circuits may provide dynamic flexing and increased heat dissipation and may be used in the embodiments that require circuits with smaller footprint, increased package density, more tolerance to vibrations, and/or less weight.

As used in this specification, the terms “computer,” “server,” “processor,” and “memory” all refer to electronic or other technological devices. These terms exclude people or groups of people. For the purposes of the specification, the terms display or displaying means displaying on an electronic device. As used in this specification, the terms “computer readable medium,” “computer readable media,” and “machine readable medium” are entirely restricted to non-transitory, tangible, physical objects that store information in a form that is readable by a computer. These terms exclude any wireless signals, wired download signals, and any other ephemeral or transitory signals.

The above description presents the best mode contemplated for carrying out the present embodiments, and of the manner and process of practicing them, in such full, clear, concise, and exact terms as to enable any person skilled in the art to which they pertain to practice these embodiments. The present embodiments are, however, susceptible to modifications and alternate constructions from those discussed above that are fully equivalent. Consequently, the present invention is not limited to the particular embodiments disclosed. On the contrary, the present invention covers all modifications and alternate constructions coming within the spirit and scope of the present disclosure. For example, the steps in the processes described herein need not be performed in the same order as they have been presented, and may be performed in any order(s). Further, steps that have been presented as being performed separately may in alternative embodiments be performed concurrently. Likewise, steps that have been presented as being performed concurrently may in alternative embodiments be performed separately.

What is claimed is:

1. A personal health monitoring device, comprising:
a plurality of electrochemical sensors, each electrochemical sensor configured to measure an electrical current in a liquid in response to receiving a voltage;
a set of one or more potentiostats, each potentiostat connected to one or more sensors in the plurality of electrochemical sensors by a plurality of wires;
a processor communicatively coupled to the set of potentiostats, wherein the processor and the set of potentiostats are configured to attach to an outside of a urine collection basin, and wherein the plurality of sensors are configured to hang from the corresponding plurality of wires and come in contact with liquid inside the urine collection basin;
wherein each potentiostat is configured to:
receive one or more signals identifying a range of voltages to apply to a set of one or more electrochemical sensors in the plurality of electrochemical sensors;
apply the identified range of voltages to each sensor;
for each applied voltage in the range of voltages, receive a current measurement for each electrochemical sensor in the set of electrochemical sensors;
send applied voltages values and current measurement values to the processor;
wherein the processor is configured to:
send one or more signals to each potentiostat identifying a range of voltages to apply to a set of one or more electrochemical sensors in the plurality of electrochemical sensors;
receive the applied voltages values and the current measurement values from the set of potentiostats; and
store the applied voltages values and the current measurement values.

2. The personal health monitoring device of claim 1 further comprising:
a temperature sensor connected to the processor by a plurality of wires and configured to come in contact with the liquid inside the urine collection basin;
wherein the processor is configured to:
receive temperature measurements from the temperature sensor; and
send the one or more signals to the set of potentiostat when the temperature of the liquid in the urine collection basin is increased by a first threshold within a predetermined time period.

3. The personal health monitoring device of claim 1, wherein the processor is configured to:
receive a set of labels comprising one or more of an identification of a person using the urine collection basin, an identification of a set of one or more conditions that are going to be predicted, and an identification of a set of one or more conditions that are going to be tracked; and
correlate the applied voltage values and the current measurement values received from the potentiostats to the set of labels.

4. The personal health monitoring device of claim 3, wherein the processor is configured to receive the set of labels from a client device associated with the person using the urine collection basin.

5. The personal health monitoring device of claim 3 further comprising a microphone, wherein the processor is configured to receive the identification of the person using the urine collection basin as a voice message captured by the microphone.

6. The personal health monitoring device of claim 3 further comprising a fingerprint reader, wherein the processor is configured to receive the identification of the person using the urine collection basin as a fingerprint reading from the fingerprint reader.

7. The personal health monitoring device of claim 1, wherein the processor is configured to identify one or more biomarkers indicating a presence and an amount of one or more metabolites in the urine.

8. The personal health monitoring device of claim 7, wherein the processor is configured to generate an alert when an amount of a metabolite in the urine is not within a predetermined range.

9. The personal health monitoring device of claim 7, wherein the processor is configured to determine health and diet trends based on changes in the amount of a metabolite in the urine.

10. The personal health monitoring device of claim 1, wherein the processor is configured to extract biological data, provide personalized health insights, detect irregularities related to human conditions, and provide early diagnosis by analyzing the voltage values and the current measurement values received from the plurality of electrochemical sensors.

11. The personal health monitoring device of claim 1, wherein the processor is configured to quantitatively determine a concentration of an metabolite of interest by comparing a peak current at a particular voltage with peak currents of a set of reference solutions which are used to precalibrate the personal health monitoring device.

12. The personal health monitoring device of claim 1, wherein the plurality of electrochemical sensors comprises a pH sensor,
wherein the processor is configured to:
receive the voltage values applied to the pH sensor and the corresponding current measurement values from a potentiostat in the set of potentiostats; and
convert the current measurements into pH values of the liquid in the urine collection basin.

13. The personal health monitoring device of claim 1, wherein each of the plurality of electrochemical sensors comprises a plurality of electrodes, wherein the plurality of electrodes of each electrochemical sensor comprises one of platinum (Pt) electrodes, gold (Au) electrodes, silver (Ag) electrodes, glassy carbon electrodes, and amorphous carbon electrodes.

14. The personal health monitoring device of claim 1, wherein each of the plurality of electrochemical sensors comprises a plurality of electrodes, wherein the plurality of electrodes of at least one electrochemical sensor comprises screen-printed electrodes (SPE).

15. The personal health monitoring device of claim 1, wherein plurality of electrochemical sensors comprises one or more of a single-walled carbon nanotubes (SWCNT) sensor, a multi-walled carbon nanotubes (MWCNT) sensor, a NiOx (Nickel Oxide based materials) screen-printed electrodes (SPEs) sensor, a sodium ion selective electrochemical sensor, a potassium ion selective electrochemical sensor, and a chloride ion selective electrochemical sensor.

16. The personal health monitoring device of claim 1, wherein each of the plurality of electrochemical sensors comprises a plurality of electrodes, wherein the plurality of electrodes of at least one electrochemical sensor in the plurality of electrochemical sensors comprises a working electrode, a reference electrode, and an auxiliary electrode, and wherein the at least one electrochemical sensor is configured to measure a current value at the auxiliary electrode in response to receiving a voltage between the working electrode and the reference electrode.

17. The personal health monitoring device of claim 1, wherein each of the plurality of electrochemical sensors comprises a plurality of electrodes, wherein the plurality of electrodes of at least one electrochemical sensor in the plurality of electrochemical sensors comprises a working electrode and an auxiliary electrode, and wherein the at least one electrochemical sensor is configured to measure a current value flowing between the working electrode and the auxiliary electrode in response to receiving a voltage between the working electrode and the auxiliary electrode.

18. The personal health monitoring device of claim 1, wherein the processor is configured to:
receive the applied voltage values and the current measurement values collected from urine of a plurality of persons using the urine collection basin;
receive a set of labels comprising an identification of a set of one or more conditions that are being tracked;
correlate the values of the applied voltages and the values current measurements to the set of labels;
identify a set of one or more features from the applied voltage values and the current measurement values, each feature associated with a range of voltage values and the corresponding current measurement values measured by an electrochemical sensor; and
determine features that are common for the set of conditions that are being tracked.

19. The personal health monitoring device of claim 1, wherein the conditions that are being tracked comprise one or more of an illness, exposure to more or more hazardous material, gender, age, diet, daily routine, sleep patterns, exercise, hormone cycles, pregnancy, biomarker changes due to development of diseases, presence of pathogens, drug use, temperature changes, and pH changes.

20. The personal health monitoring device of claim 1, wherein the processor is configured to:
receive an identity of a set of one or more features, each feature corresponding to a metabolite, each feature identified by a range of applied voltage values and the corresponding current measurement values; and
compare the applied voltage values and the current measurement values received from the set of potentiostats with the range of applied voltage values and the corresponding current measurement values of each feature; and
determine an existence of one or more of the features in the applied voltage values and the current measurement values received from the set of potentiostats based on the comparison.
